(19) United States
(12) Patent Application Publication
(10) Pub. No.: US 2021/0252217 A1
(43) Pub. Date: Aug. 19, 2021

(54) THERAPEUTIC INFUSION NEEDLE

(71) Applicant: Cook Regentec LLC, Indianapolis, IN (US)

(72) Inventors: Frank J. Fischer, JR., Bloomington, IN (US); Jonathan Lee Bennett, Bloomington, IN (US); Michael P. DeBruyne, Bloomington, IN (US)

(21) Appl. No.: 17/313,857

(22) Filed: May 6, 2021

Related U.S. Application Data
(63) Continuation-in-part of application No. PCT/US2019/060053, filed on Nov. 6, 2019.
(60) Provisional application No. 62/756,314, filed on Nov. 6, 2018.
(60) Provisional application No. 62/814,908, filed on Mar. 7, 2019.

(51) Int. Cl. A61M 5/162

(52) U.S. Cl. CPC A61M 5/1626; A61M 2205/82; A61M 2205/3344

(57) ABSTRACT

Devices and methods useful for injecting therapeutics into the body of a patient are illustrated and described herein. In certain aspects, needle assemblies comprising a needle having a needle shaft with one or more side openings and a needle sheath slidably moveable along the needle shaft to selectively expose or cover all or a portion of the one or more side openings are provided. Such assemblies may include an interference fit between the needle sheath and the needle shaft and/or a seal positioned around the needle shaft proximal of the plurality of one or more side openings and configured to prevent fluid from leaking between the needle sheath and the needle shaft and beyond a proximal end of the needle sheath.

### FIELD OF THE INVENTION

The present disclosure pertains generally to devices and methods useful for injecting therapeutic agents into the body of a patient.

### BACKGROUND OF THE INVENTION

Some medical therapies deliver therapeutic agents that negatively impact both to healthy/normal cells and unhealthy/abnormal cells. In chemotherapy, for example, delivered agents affect both normal and abnormal cells of the patient. Similarly, should a medical professional providing treatment to the patient come into contact with a therapeutic agent, the agent can negatively affect the normal cells of the medical professional.

Moreover, due to the costs and complexities in manufacturing certain therapeutic agents, some therapies use only a small volume of therapeutic agent. For example, in T-cell immunotherapy, T-cells are removed from a patient and modified in a laboratory to target and eliminate cancer cells. Those modified cells are then multiplied in the laboratory and returned to the patient to attack the cancer cells in the patient's body. As the modified cells are specific to the patient and expensive and difficult to manufacture, only small volumes are often formed.

While devices for the delivery of therapeutic agents are generally known, there remains a desire for improved and new devices and methods, particularly in view of the above-identified considerations.

### SUMMARY

The present disclosure pertains generally to devices and methods useful for injecting therapeutics into the body of a patient. In certain aspects, the present disclosure provides needle assemblies having one or more side openings that can be selectively closed and/or opened for the delivery of therapeutics, such as by a needle sheath slidably positioned around a needle shaft and configurable from a first configuration in which the needle sheath sealingly covers one or more side openings to a second configuration in which the one or more side opening is/are at least partially uncovered by the needle sheath. For example, the present disclosure provides needle assemblies comprising a needle having a needle shaft with side openings and a needle sheath to slidably moveable along the needle shaft to selectively expose or cover all or fewer than all of the side openings.

Devices and methods of the present disclosure are arranged to reduce the risk of exposure of therapeutic agents to normal tissue and/or to a medical professional. Preferably, introduction of a therapeutic agent into a patient is limited to only the abnormal cells (e.g., the tumor) of the patient. Moreover, as certain therapies use only small volumes of therapeutic agent, there is a need for devices and methods that maximize the volume of therapeutic agent delivered into the target area.

Embodiments of needle assemblies described herein can comprise: a needle defining a lumen, the needle having a needle shaft extending from a hub; the needle shaft having a proximal portion and a distal portion; the distal portion having a distal tip and one or more side openings; a needle sheath slidably positioned around the needle shaft, wherein the needle sheath is configurable from a first configuration in which the needle sheath sealingly covers the side opening to a second configuration in which the side opening is at least partially uncovered by the needle sheath (e.g., is partially or fully uncovered by the needle sheath).

The needle sheath can have at least a portion that has an interference fit with the needle shaft. The needle assembly can include a seal positioned around the needle shaft proximal of the side opening and configured to prevent fluid from leaking between the needle sheath and the needle shaft and beyond a proximal end of the needle sheath. A distal end of the needle sheath may also have an interference fit with the needle shaft to seal a distal end of a lumen defined by the needle sheath.

The needle sheath can be effective to prevent a fluid leak with a static fluid pressure of at least 50 mmHg, at least 250 mmHg, at least 500 mmHg, at least 750 mmHg, and/or at least 1000 mmHg in the lumen when the needle sheath covers the side opening and the needle assembly is in 1 atm. For example, such arrangements may prevent a fluid leak from the side opening and/or from between the needle sheath and the needle shaft and beyond a proximal end of the needle sheath and/or beyond a distal end of the needle sheath. Any of the needle assemblies disclosed herein may be included in a kit comprising a therapeutic agent suspended in a volume of liquid.

Exemplary methods of infusing a therapeutic agent into a volume of tissue, comprise: i. inserting a distal portion of a needle assembly of the present disclosure into the volume of tissue in a first configuration, such as a needle assembly comprising a needle shaft and a needle sheath, the needle shaft having a length, a distal tip, and a side opening in the distal portion, the needle sheath slidably positioned around the needle shaft, the needle sheath configurable from the first configuration in which the needle sheath sealingly covers the side opening to a second configuration in which the side opening is at least partially uncovered; ii. configuring the needle assembly into the second configuration within the volume of tissue; iii. infusing a therapeutic through the needle shaft in the second configuration and out of the exposed side opening into the volume of tissue; iv. configuring the needle assembly into the first configuration within the volume of tissue by withdrawing the needle shaft relative to the needle sheath such that the needle shaft moves a greater distance relative to the volume of tissue than the needle sheath; and v. retracting the needle shaft in the first configuration from the volume of tissue.

Needle assemblies of the present disclosure comprise a needle and a needle sheath. The needle can be one useful for intratumoral, intraorgan (e.g., liver, kidney), intramuscular, subcutaneous, intracutanous, subdermal, and/or intradermal infusion of diagnostic and therapeutic agents. The needle includes a needle shaft and a hub. The needle shaft is a hollow, elongate member extending from the hub. The needle shaft has a proximal portion and a distal portion and a sidewall defining a lumen. The proximal portion is affixed to the hub. The distal portion of the needle shaft has a distal tip and a side opening in communication with a lumen of the needle. The side opening extends from an inner surface of the sidewall facing the lumen to an outer surface of the sidewall. The lumen is preferably free of a movable tube positioned therein.

The needle can have a needle shaft having a length of at least 5 cm, at least 7.5 cm, at least 10 cm, or at least 15 cm. The needle (e.g., a catheter needle) may have needle shaft having a length of at least 30 cm, at least 35 cm, at least 40 cm, or 45 cm. The needle (e.g., catheter needle) may even have a needle shaft having a length of at least 100 cm or at least 200 cm (e.g., 220 cm or 230 cm). It is envisioned that any of the needles and/or needle shafts disclosed herein may have a length sufficient for use with an endoscope (e.g., an endoscope for fine needle biopsy). The endoscope may be a bronchoscope, colonoscope, cytoscope, duodenoscope, enteroscope, gastroscope, laparoscope, laryngoscope, thoracoscope, or ureteroscope, just to name a few non-limiting examples. The needle shaft of any needle disclosed herein may have a needle gauge of at least 14 and up to 32. Preferably, the needle gauge is at least 19, at least 21, at least 23, or at least 25.

The distal tip can be a non-coring tissue penetrating needle tip. The distal tip can be an open-tip or a closed-tip (i.e., free of an opening for fluid flow). For example, the distal tip can be a trocar tip, such as a triple bevel trocar. The needle may or may not be tapered.

The needle may include echogenicity-enhancing features, including but not limited to dimples and/or grooves. Such features can be on the outer surface of the needle.

The side opening(s) can be spread longitudinally along a length of the distal portion and define an infusion section. The infusion section may have a length along a longitudinal axis of the distal portion that is up to 50% of the length of the needle shaft, up to 20% of the length of the needle shaft, and/or at least 5% of the length of the needle shaft. The infusion section may have a length along a longitudinal axis of the distal portion of up to 1 cm, 1 cm to 2 cm, or up to 2 cm, as just a few non-limiting examples.

The side opening(s) are preferably spread around the circumference of the distal portion. For example, side opening(s) can be spread helically around a longitudinal axis of the distal portion. As another example, side openings can be arranged in rings that extend circumferentially around the needle shaft—the rings being parallel to one another and spaced longitudinally along a length of the distal portion. In at least one embodiment, the side openings define an infusion segment of the distal portion and are positioned helically around a longitudinal axis of the distal portion to uniformly deliver therapeutic agents during use. The helixes and/or rings may be arranged in at least 4 revolutions around the needle per linear centimeter of the needle. Preferably, the helixes and/or rings are arranged in at least 6 revolutions per linear centimeter of the needle.

Side openings may be spaced apart along the infusion section to have a longitudinal distance between adjacent side openings. For example, a distal-most edge of a first side opening may be longitudinally spaced proximally of a proximal-most edge of a second side opening adjacent-to and positioned distally of the first side opening. Alternatively or additionally, side openings may at least partially overlap one another along the infusion section such that a distal-most edge of first side opening is positioned distally of a proximal-most edge of a second side opening adjacent to and positioned distally of the first side opening.

The needle may have side holes in a concentration of at least 10 holes per linear centimeter of the needle. Preferably, the needle has side holes in a concentration of at least 20 holes per linear centimeter and, more preferably 22 holes per linear centimeter. The concentration of side openings may vary along the distal portion. For example, more side openings may be positioned near the distal tip than near a proximal end of the distal portion or vice versa. Such an arrangement can be achieved by having less spacing between side openings and/or greater overlap of side openings in one region of the distal portion than another region. For example, side openings may be arranged along a helix extending around the longitudinal axis of the distal portion, wherein the helix has a greater helix angle near the distal tip than near the proximal end of the distal portion or vice versa.

The side openings preferably have a cross-sectional dimension (measured at an inside surface of the needle) of at least 60% of an inside diameter of the needle. More preferably, the cross-sectional dimension is at least 70%, 80%, or 90% of the inside diameter of the needle. The cross-sectional dimension of the side openings at an inside surface of the needle may be less than a cross-sectional dimension of the side openings at an outside surface of the needle due to the wall thickness of the needle. The cross-sectional area of the side opening(s) may vary along the distal portion. For example, side openings positioned nearer the distal tip may have a larger cross-sectional area than side openings positioned nearer the proximal end or vice versa.

For a length of the needle measured between side openings, the percentage of surface area of an inner surface of the needle occupied by side openings may be at least 20%. Preferably, the inner surface area occupied by side openings is less than 30%. More preferably, the inner surface area occupied by side openings is 25%.

The side openings may have a circular or non-circular cross-sectional shape. Preferably the side opening shape is that of a circle wrapped onto the outer surface of the needle. The preferred orientation of the circle is perpendicular to a longitudinal axis of the needle. Side openings of different cross-sectional areas may be achieved by having side openings that are more elongate than other side openings. For example, side openings nearer the distal tip may be more elongate than more proximally-located side openings or vice versa.

A width of an elongate side opening, as measured around the circumference of the needle shaft, may vary along a length of the elongate side opening. For example, a distal portion of the elongate side opening may have a greater width than a proximal portion of the elongate side opening or vice versa.

The side opening(s) can have any of a variety of cross-sectional shapes. The side opening(s), for example, can have a circular or oval cross-sectional shape. Elongate cross-sectional shapes, such as ovals, can have a longitudinal axis aligned parallel with the longitudinal axis of distal portion, transverse to the longitudinal axis of the distal portion, or between the two.

In an exemplary embodiment, side openings have the shape of a circle wrapped onto the outer surface of the needle. The orientation of the circle is perpendicular to a longitudinal axis of the needle. The diameter of each side opening (measured at the inside surface of the needle) is 80% of the inside diameter of the needle. There are at least 20 side openings per linear centimeter of the needle. The side openings occupy at least 20% of the surface area of the inner surface of the needle. The side openings are arranged helically around the needle in 5 to 7 revolutions per linear centimeter.

The needle may be a metallic hypo-tube. The side openings may be laser cut into the needle. Edges of the side openings at an outer surface of the needle are preferably radiused. Such radius may be formed by electropolishing.

The proximal portion of the needle shaft may include indicia spread along a length thereof. As will be explained below, such indicia is indicative of the number and/or area of exposed side opening(s) and/or length of the distal portion of the needle shaft with exposed side opening(s) when a portion of the sheath is aligned with the indicia. The indicia will most often be visualizable indicia, such as graduated markings; however, other indicia are envisioned. For example, the indicia may include tactile and/or audible signals (e.g., clicks) that are detectable upon movement of the sheath relative to the needle shaft.

The needle sheath may include indicia useful for measuring a dimension of the target area. For example, the needle sheath may include indicia indicating the distance from the indicia to a distal-most end of the needle sheath and/or to the distal tip of the needle shaft. The needle sheath may include one or more markers for visualization under medical imaging equipment (e.g., radiography and/or ultrasound). For example, the needle may include a radiographic marker (e.g., a metal band) and/or an echogenic feature (e.g., dimples) at a distal-most end of the needle sheath.

The needle shaft may have a first outer diameter along a first length and a second outer diameter along a second length, the first outer diameter being greater than the second outer diameter. Preferably the first outer diameter is at least 10% greater than the second outer diameter. More preferably, the first outer diameter is at least 20% greater than the second outer diameter. The first length is preferably proximal of the second length—the second length being proximal of a tissue-penetrating needle tip. The wall thickness of the needle shaft (measured between an inner surface and an outer surface of the needle shaft) may be greater in the first length than in the second length. The needle shaft may include an outer sleeve positioned around an inner needle and along said first length. Preferably, the outer sleeve is fixed relative to the inner needle (e.g., longitudinally and/or rotationally fixed relative thereto). For example, the outer sleeve may be soldered to the inner needle. The needle shaft may also be formed by drawing down a needle to form the different diameters of the first and second lengths. Additionally or alternatively to any of the embodiments disclosed herein, the needle shaft may be subjected to grinding to form, at least partially, the first and/or second outer diameters and/or the distal tip of the needle shaft.

The hub of the needle can include one or more connectors, such as a luer and/or tuohy borst connector for fluidly connecting the lumen of the needle with another device (e.g., a pressure monitor and/or syringe). The hub may include a valve, such as a tuohy borst valve, arranged to close the fluid flow-path in the hub extending towards the needle lumen.

The needle sheath extends along an outer surface of the needle shaft. The needle sheath may be a catheter. The needle sheath has a length less than that of the needle shaft. For example, the needle sheath may be shorter than the length of the needle shaft by 20 cm or less, shorter than the length of the needle shaft by 10 cm or less, or shorter than the length of the needle shaft by 5 cm or less. Preferably, the needle sheath has a length no greater than the distance from a proximal end of the needle shaft to the proximal-most side opening of the distal end of the needle. Preferably, the needle sheath has a length less than a distance between an indicia on the needle shaft and the distal tip. Such indicia may be a distal-most indicia on the needle shaft.

The needle sheath has an inner surface defining a lumen for receiving the needle shaft. At least a portion (e.g., a distal portion) of the lumen defined by the needle sheath can have a cross-sectional dimension smaller than a cross-sectional dimension of the needle shaft to form an interference fit between the needle sheath and the needle shaft when the needle shaft is received within the lumen of the needle sheath. The inner surface of the needle sheath can taper inwardly (i.e., toward the lumen) along a length of the needle sheath. For example, the inner surface may taper inwardly along a proximal-to-distal direction along a portion of the needle sheath (e.g., a distal portion). Such an arrangement can form an interference fit between the distal end of the needle sheath and the needle shaft. Preferably, the outer surface of the needle sheath tapers inwardly (i.e., towards the needle sheath lumen). Most preferably, when the needle sheath is positioned around the needle shaft, the transition from the needle shaft to the distal tip of the needle sheath is free of an exposed edge that could contact patient tissue during insertion of the needle assembly into patient tissue.

The needle assembly can have a seal arranged to resist fluid from passing between the needle sheath and the needle. For example, the needle sheath may include an o-ring, a septum, and/or a valve (e.g., a tuohy borst valve) that surrounds and contacts an outer surface of the needle shaft. The seal may be coupled to the sheath such that the seal moves with the sheath along the needle shaft. Alternatively, the seal may be coupled to the needle shaft such that movement of the sheath along the needle shaft also moves the sheath along the seal.

Preferably, the needle sheath is effective to prevent fluid from leaking between the needle sheath and the needle shaft with a static fluid pressure of at least 50 mmHg in the lumen when the side openings are covered by the needle sheath and the needle assembly is in 1 atm. More preferably, the needle sheath is effective to prevent fluid from leaking between the needle sheath and the needle shaft with a static fluid pressure of at least 250 mmHg in the lumen when the side openings are covered by the needle sheath and the needle assembly is in 1 atm.

The needle sheath can include a fluid port (e.g., a male or female luer) allowing access to the lumen defined by the needle sheath when the needle sheath is positioned around the needle shaft. Such a fluid port may allow for fluid (e.g., saline) to be positioned into the lumen defined by the needle sheath and around the needle shaft. The fluid port may include a valve. Having fluid positioned in the needle sheath lumen around the needle shaft during an infusion can resist therapeutic agent from exiting the needle shaft (e.g., from the proximal or distal ends of the needle sheath). Fluid in the needle sheath lumen may also resist therapeutic agent from exiting a side opening of the needle shaft and entering the lumen of the needle sheath (for any side openings positioned within the needle sheath). Advantageously, this can increase the volume of therapeutic agent delivered to the target tissue for a given volume of therapeutic agent introduced into the proximal end of the needle shaft. The fluid port may also be used to flush therapeutic agent from the side openings of the needle shaft when the side openings are within the needle sheath, with the flush fluid passing through the side openings of the needle from the lumen of the to needle sheath towards the lumen of the needle shaft.

The needle assembly may include a pressure monitor coupled to a proximal end of the needle. The pressure monitor is preferably arranged to measure the pressure (static and/or dynamic) of therapeutic being introduced into the needle. The pressure monitor may be a disposable pressure transducer, such as the pressure transducer marketed under the name Compass® CT by Cook Regentec of Indianapolis, Ind., USA.

The needle assembly may also include an injector for injecting therapeutic into the pressure monitor and/or the needle. The injector may comprise a syringe and/or a power injector. The injector may be coupled to the needle via the pressure monitor, the injector and pressure monitor may each be coupled to the needle, or the needle or injector may include the pressure monitor, just to name a few non-limiting examples.

An injector and, optionally, a pressure monitor, such as any of those discussed above, may be coupled to the needle sheath for injecting fluid into the lumen of the needle sheath.

The needle assembly may include an RF power supply for supplying RF power to the needle shaft. Similarly, the needle assembly may include a return electrode connected to the RF power supply and arranged to complete a circuit extending from the RF power supply to the needle shaft, from the needle shaft to patient tissue, from patient tissue to the return electrode, and from the return electrode to the RF power supply.

The needle assembly may include a spacer that retains the needle sheath in position relative to the needle shaft, and vice versa. Advantageously, the spacer may aid in maintaining position of the needle sheath relative to the needle shaft during insertion and/or retraction of the needle assembly in patient tissue (e.g., for maintaining the needle sheath over the side openings of the needle shaft). For example, the spacer may resist withdrawal of the needle sheath relative to the needle shaft when attached to the needle sheath and/or needle shaft.

The needle assembly may also include a lock for securing the needle sheath to the needle shaft at a desired location (e.g., resisting movement of the needle sheath along the needle shaft). The lock, when engaged, may apply a compressive force to a portion of the needle shaft. The lock may include a clamp, a setscrew, and/or a valve (e.g., a Touhy Borst valve), just to name a few non-limiting examples. For example, the clamp lock may include the device of U.S. Pat. No. 4,453,292, or the device of U.S. Pat. No. 8,647,261, each of which incorporated by reference. Preferably, the lock is arranged for operation without requiring any further tools or accessories outside of the needle assembly. For example, the lock may include a handle and/or button to facilitate engagement/disengagement of the lock using a physician's fingers.

The needle assembly may include a threaded needle shaft and/or needle sheath to allow more precise movement of the needle sheath to relative to the needle hub. For example, the needle sheath may include a rack and/or threaded portion (e.g., a threaded sleeve positioned over the needle sheath), and the hub of the needle sheath may include a free-floating nut that, when rotated, advances the needle shaft distally or proximally relative to the needle sheath. Additionally, the rack/threaded needle shaft and/or needle sheath may resist longitudinal movement of the needle sheath relative to the needle shaft due to the resistance of patient tissue during insertion and/or withdrawal of the needle assembly in/from a patient (e.g., resists back-driving of the free-floating nut).

### Methods of Use

Prior to insertion into the patient, the needle sheath may be retracted along the needle shaft to have one or more side openings (e.g., of a plurality of side openings) exposed in the distal portion of the needle shaft. Such an arrangement may be desirable to facilitate flushing of the needle lumen prior to insertion into the patient.

Prior to inserting the needle into tissue of the patient, the needle sheath may be advanced distally relative to the needle shaft to cover the one or more side openings of the needle shaft. With the needle sheath covering the one or more side openings of the needle shaft, the needle sheath and needle shaft are advanced together into and/or through the tissue of the patient and towards a target area. Preferably none of the side openings are exposed outside of the needle sheath during advancement into (i.e., insertion) and/or through the tissue of the patient towards the target area.

The needle shaft and needle sheath are preferably advanced through the target area together (e.g., with the needle sheath covering the side openings of the needle shaft) until the distal tip of the needle shaft is adjacent the periphery of the target area (e.g., the boundary of the abnormal tissue and adjacent normal tissue). Preferably, the needle sheath covers the side openings of the needle shaft during advancement of the needle assembly into the patient to resist clogging of the side openings by patient tissue. Such positioning may locate at least the distal tip of the needle shaft within the target area. For example, the distal-most side opening and, at least a portion of the one or more side openings, may be located within the target area.

After the distal tip of the needle shaft is adjacent to the periphery of the target area (e.g., a tumor), the needle shaft may be advanced distally relative to the needle sheath to expose one or more side openings of the needle shaft in the target area. This may be accomplished, for example, by advancing the needle shaft through the target area while retaining the needle sheath in a stationary position or advancing both the needle sheath and the needle shaft through at least a portion of the target area and then withdrawing the needle sheath while retaining the needle shaft in a stationary position.

Preferably, after advancing the needle shaft and/or needle sheath into the target area, the needle shaft and/or needle sheath span at least 80% of the target area along a longitudinal axis of the needle. More preferably, the needle shaft and/or needle sheath span at least 80% of the target area along a longitudinal axis of the needle. In some instances, the needle shaft and/or needle sheath span at least 90% of the target area along a longitudinal axis of the needle.

After the needle sheath is withdrawn relative to the needle shaft (e.g., holding the needle sheath stationary and advancing the needle shaft or holding the needle shaft stationary and retracting the needle sheath) one or more side openings positioned within the target area are exposed. For example, in instances where the needle shaft is held in a stationary location and the needle sheath is retracted, the needle sheath may be withdrawn relative to the needle shaft until the distal-most end of the needle sheath is positioned adjacent a peripheral edge of the target area. Alternatively, in instances where the needle sheath is held stationary and the needle shaft is advanced distally, the needle shaft may be advanced until the distal tip is adjacent a periphery of the target area opposing the periphery of the target area adjacent the distal end of the needle sheath. Preferably, a plurality of side openings are exposed in the target area. More preferably, all of the side openings of the needle shaft are exposed in the target area.

Preferably, the distal tip of the needle shaft does not pass beyond the target area to exit the target area and enter healthy tissue. Keeping the needle shaft distal tip within the target area is believed to aid in keeping more therapeutic agent within the target area. Advantageously, this can aid in a more efficient usage of the therapeutic agent in treating the target area. But, significantly, this can also reduce the contact of therapeutic agent with healthy tissue outside of the target area, which may have a detrimental effect to such healthy tissue.

As a medical professional may not be able to visualize the one or more side openings to being exposed inside the target area, the needle shaft may be withdrawn until a portion of the needle sheath aligns with a predetermined indicia on the needle shaft. Such predetermined indicia may be determined by a prior measurement of the target area along the direction of insertion of the needle assembly into the target area. Such prior measurement may been made under x-ray or ultrasound imaging. The prior measurement may have been made after the needle assembly was positioned within the target area, such as by the alignment of the patient's skin with indicia on the needle sheath indicating the length of the needle assembly positioned within the patient's tissue. As will be appreciated by those of ordinary skill in the art, such measurement may be adjusted by the distance from the outer surface of the patient's skin to the target area, as measured along the needle assembly.

With the needle assembly in position and the desired area and at least a portion of the one or more side openings exposed within the target tissue, therapeutic agent may be infused through the lumen of the needle and out of the exposed one or more side openings into the target area.

The target area tissue may heated prior to, during, and/or after infusion of therapeutic agent. For example, RF energy may be applied by an RF power supply to the target area through use of one or more electrodes (e.g., the needle shaft and a surface electrode on the skin of the patient). Preferably, the target area tissue is heated to a temperature of less than 45° Celsius to avoid ablation of target area tissue. It is believed that heating the target area tissue to a temperature of up-to but less than 45° Celsius can increase the cellular adhesion and/or uptake of therapeutic agent delivered into the target area.

After delivery of therapeutic agent and/or heating the target area, the needle shaft can be retracted relative to the needle sheath and/or the needle sheath may be advanced relative to the needle shaft to sealingly cover and close the one or more side openings. During such retraction and/or advancement, the needle sheath or the needle shaft may move, relative to the target area, a distance greater than that of the other. Additionally or alternatively, the needle sheath or the needle shaft may move less than 10% of the length of the needle shaft or a distance less than 10% of the length of the needle shaft relative to the target area. For example, the needle sheath may be retained in position relative to the target area and the needle shaft retracted into the needle sheath to cover the side openings. Preferably, the side openings are sealingly covered without exposing the side openings outside of the target area.

Covering the one or more side openings of the needle shaft with the needle sheath prior to retracting the needle assembly out of the target area can contain needle contents (e.g., therapeutic agent) during withdrawal and disposal of the needle. Advantageously, such an arrangement can reduce the likelihood of exposure of therapeutic agent to tissue outside of the target area (e.g., normal tissue). Additionally, covering the one or more side openings prior to withdrawal of the needle assembly can reduce the likelihood of exposure of medical personnel to therapeutic agent. It is believed that withdrawing the needle shaft from the target area into the needle sheath prior to overall withdrawal of the needle assembly from the patient may allow for closure of the needle tract created by the needle shaft. Advantageously, allowing the needle tract in the target tissue to close prior to withdrawing of the needle assembly from the target area can reduce the likelihood of injected therapeutic agent leaking out of the target area through the needle tract. In some instances, an operator may wait a period of time (e.g., one to five minutes) after retracting the needle shaft into the needle sheath before retracting the needle assembly to allow the needle track within the target area tissue to close. It is believed the distal tip of the needle shaft and/or needle sheath may aid in closing (e.g., plugging) an end of the tract in the target area and resisting leakage of therapeutic agent therefrom. For example, the needle sheath is believed to extend radially further from the needle shaft than the tract in patient tissue formed by the needle shaft and, therefore, block an end of the tract. It is also believed that allowing the needle tract to close prior to retracting the needle assembly can provide further uptake of therapeutic agent into tissue of the target area.

As mentioned above, alternatively to inserting the needle assembly (e.g., both the needle shaft and the needle sheath) across the target area and then retracting the needle sheath relative to the needle shaft to expose one or more side openings within the target area, the needle shaft may be advanced distally beyond a distal end of the needle sheath to expose one or more side openings within the target area. For example, the needle assembly may be advanced towards the target area until a distal-most end of the needle sheath is at a periphery of the target area. Then, the needle shaft may then be advanced further, relative to the needle sheath, to span the target area. In such an instance, the needle sheath may be held in position while the needle shaft is advanced.

Advantageously, in either method of exposing the one or more side openings within the target area (e.g., withdrawing the sheath or advancing the needle shaft), the one or more side openings are covered during advancement of the needle assembly towards the target area and withdrawal of the needle assembly from the patient after infusion.

The needle sheath of the present disclosure is believed to aid in resisting therapeutic agent from passing proximally through the needle tract, along the needle shaft, during and/or after infusion. As the needle sheath is larger in cross-sectional dimension (e.g., diameter) than the needle shaft, the distal-most end of the needle sheath is believed to resist therapeutic agent from traveling proximally along the needle shaft through the tract.

In any of the embodiments disclosed herein, the needle shaft may also have a first outer diameter along a first length and a second outer diameter along a second length, the first outer diameter being greater than the second outer diameter. The first length may be proximal of the second length Similar to the needle sheath, discussed, above, the larger outer diameter of the first length is believed to resist therapeutic agent from traveling proximally from the second length along the needle shaft through the tract—as the outer diameter of the first length is believed to be larger than the tissue tract formed by the outer diameter of the second length.

The needle shaft may define a shoulder between the first length and the second length. The shoulder may have a surface extending transverse to an outer surface of the first length and/or second length. Preferably, the shoulder has a surface intersecting an outer surface of the first length and/or second length at an angle of at least 10°, at least 20°, or at least 30°.

The shoulder is believed to resist therapeutic agent from passing from the second length to the first length along the needle shaft. When positioned within patient tissue, the shoulder can extend radially away from the longitudinal axis of the needle a distance further than the tissue tract formed by the length of needle distal of the shoulder (e.g., the second length discussed above). Therefore, the shoulder can plug an end of the needle tract. When the shoulder is positioned with in the needle sheath, the outer extent of the shoulder preferably fills the cross-sectional area of the needle sheath. And, in instances when the needle sheath is retracted such that a distal portion of the needle sheath (e.g., a tapering inwardly portion of the inner surface) lies along the shoulder, the shoulder can provide a greater surface area contacting a distal portion of the inner surface of the needle sheath to provide greater resistance to fluid flowing proximally of the shoulder between the needle shaft and the needle sheath.

Further forms, objects, features, aspects, benefits, advantages, and embodiments of the present invention will become apparent from a detailed description and drawings provided herewith.

### BRIEF DESCRIPTION OF THE DRAWINGS

FIG. 1 is a cross-sectional view of an exemplary needle assembly of the present disclosure.

FIG. 2 is a cross-sectional view of a needle sheath of a needle assembly.

FIG. 3 is another cross-sectional view of an exemplary needle assembly.

FIG. 4 is a plan view of a needle assembly without a protective cover.

FIGS. 5, 6, 7, and 8 illustrate various configurations of a needle assembly.

FIGS. 9, 10, and 11 illustrate fluid flow through a needle assembly in various configurations.

FIGS. 12, 13, 14, 15, 16, and 17 illustrate a method of using a needle assembly.

FIG. 18 illustrates a needle assembly comprising a pressure monitor and an injector.

FIG. 19 illustrates a needle assembly having an elongate side opening with a major axis extending helically around the longitudinal axis of the needle shaft.

FIG. 20 illustrates a needle assembly having an elongate side opening with a major axis extending parallel to the longitudinal axis of the needle shaft.

FIG. 21 is a cross-sectional view of another exemplary needle assembly of the present disclosure.

FIGS. 22 and 23 illustrate a method of using a needle assembly.

FIG. 24 is a side view of an exemplary needle assembly and sheath lock.

FIG. 25 is a side view of another exemplary needle assembly and sheath lock.

### DESCRIPTION OF THE SELECTED EMBODIMENTS

For the purpose of promoting an understanding of the principles of the invention, reference will now be made to the embodiments illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates. One embodiment of the invention is shown in great detail, although it will be apparent to those skilled in the relevant art that some features that are not relevant to the present invention may not be shown for the sake of clarity.

With respect to the specification and claims, it should be noted that the singular forms “a”, “an”, “the”, and the like include plural referents unless expressly discussed otherwise. As an illustration, references to “a device” or “the device” include one or more of such devices and equivalents thereof. It also should be noted that directional terms, such as “up”, “down”, “top”, “bottom”, and the like, are used herein solely for the convenience of the reader in order to aid in the reader's understanding of the illustrated embodiments, and it is not the intent that the use of these directional terms in any manner limit the described, illustrated, and/or claimed features to a specific direction and/or orientation.

The drawing figures referred to herein are provided for illustrative purposes only. They should not be construed as limiting the scope of the invention defined by the claims, including that they may not necessarily be drawn to scale. For example, the needle assembly in any of the illustrated embodiments may be a catheter needle assembly (e.g., the needle may be a catheter needle and/or the needle sheath may be a catheter)—especially one having length sufficient for use through an endoscope.

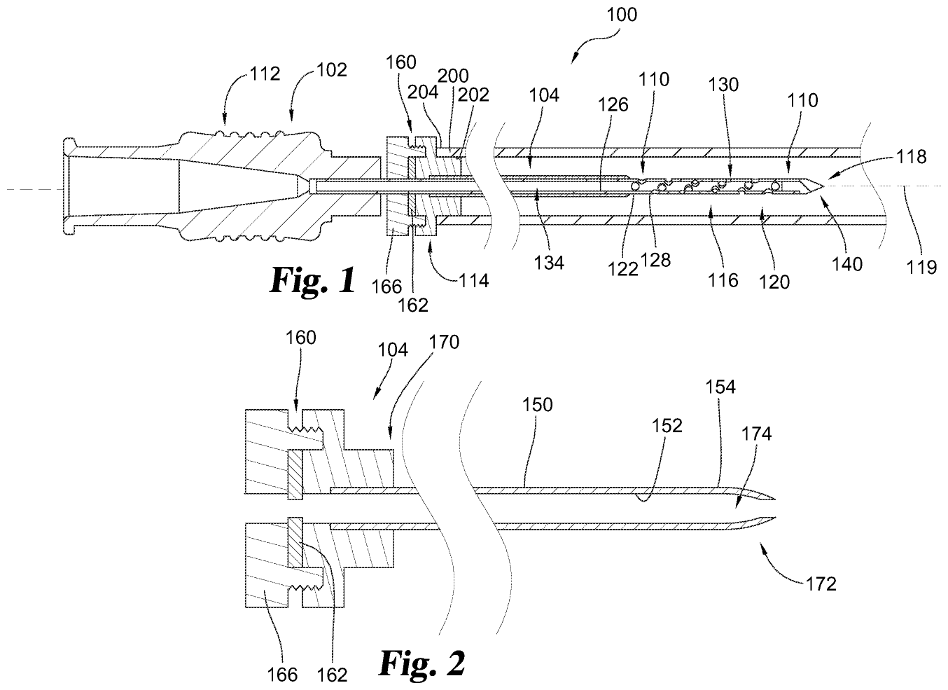

FIG. 1 illustrates a needle assembly 100. The needle assembly comprises a needle 102 and a needle sheath 104. The needle includes a needle shaft 110 and a hub 112. The needle sheath is slidably positioned along the needle shaft 110.

The needle shaft is a hollow, elongate member. The needle shaft has a proximal portion 114 and a distal portion 116. The proximal portion is affixed to the hub. The needle shaft extends distally from the hub and terminates at a distal tip 118. The needle shaft defines a longitudinal axis 119.

Positioned in the distal portion proximally of the distal tip 118 are a plurality of side openings 120 defined by a sidewall 122 of the needle shaft. The side openings 120 extend from an inner surface 126 of the sidewall to an outer surface 128 of the sidewall. The side openings define an infusion section 130. The inner surface defines a lumen 134 extending along a length of the needle shaft and in fluid communication with the hub.

The distal tip of the needle shaft may have a closed-tip. For example, the distal tip may include a trocar 140 that closes a distal-most end of the lumen of the needle shaft. The trocar may extend at least partially into the lumen. The trocar may have a triple bevel.

The needle sheath is slidable along a length of the needle shaft. The needle sheath includes a wall 150 having an inner surface 152 and an outer surface 154. As shown in FIG. 2, the needle sheath may include a seal 160. The seal extends circumferentially around the needle shaft and is arranged to resist fluid from passing between the needle shaft and the needle sheath beyond the proximal end of the needle sheath. The seal may include an O-ring 162 or a valve, such as a tuohy borst valve. The seal may be retained in association with the needle sheath by a seal housing 166 affixed to a proximal end 170 of the wall. Advantageously, the seal housing may also provide a handle for operation by a medical professional during use.

In a distal end 172 of the needle sheath, the wall may taper inwardly such that the needle sheath and/or a lumen of the needle sheath has a smaller cross-sectional dimension in the distal end than in the proximal end of the needle sheath.

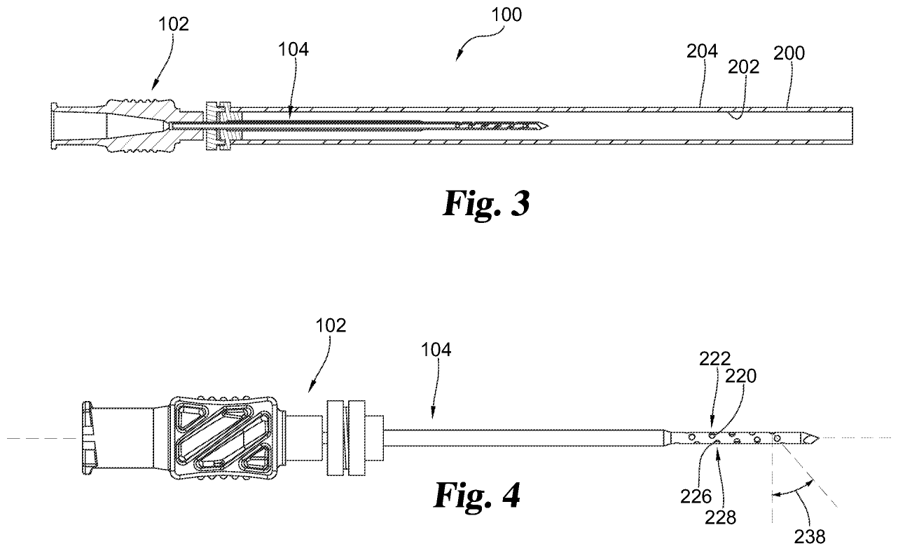

The needle assembly may include a protective cover 200, illustrated in FIGS. 1 and 3, for shipping and/or storage purposes. The protective cover may have an inner surface 202 and an outer surface 204. When assembled onto the needle, the protective cover contacts the needle hub and/or needle sheath. Preferably, the protective cover extends distally beyond the distal tip of the needle assembly to protect against unintentional needle sticks during handling.

As illustrated in FIG. 4, side openings may at least partially overlap one another along the infusion section. For example, a distal-most edge 220 of a first side opening 222 is positioned distally of a proximal-most edge 226 of a second side opening 228 adjacent to and positioned distally of the first side opening. It contemplated, however, that a distal-most edge of a first side opening may be aligned or longitudinally spaced proximally of a proximal-most edge of a second side opening adjacent-to and positioned distally of the first side opening.

Also illustrated in FIG. 4, side openings spread around the circumference of the distal portion. For example, side openings can be spread helically around a longitudinal axis of the distal portion. Such side openings may extend along a helix having a helix angle 238. While the side openings shown in FIG. 4 extend along a helix having a constant helix angle along the infusion section, it is contemplated that the helix angle may vary along a length of the infusion section.

The figures herein also illustrate a needle assembly having side openings of circular cross-sectional shape and equal cross-sectional area along the infusion section. It is contemplated, however, that side openings can have any of a variety of cross-sectional shapes and/or cross-sectional area of side openings may vary along the distal portion, as described elsewhere herein.

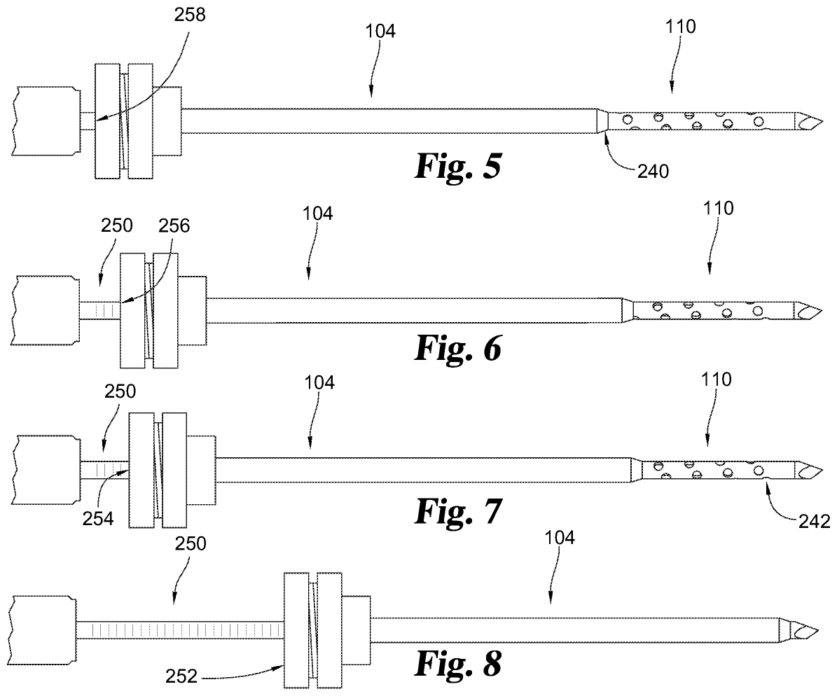

Turning now to a discussion of the operation of the needle assembly, FIGS. 5-8 show various configurations of the needle assembly. In one configuration, the needle sheath may be positioned proximally of the side openings of the needle shaft, as shown in FIG. 5. In another configuration, shown in FIGS. 6 and 7, the needle sheath may be positioned distally along the needle shaft such that one or more side openings of the plurality of side openings, such as side opening 240, are sealingly covered by the needle sheath to prevent fluid from exiting the covered side opening(s) of the needle shaft. In yet another configuration, the needle sheath extends distally beyond a distal-most side opening 242 of the plurality of side openings such that the plurality of side openings are all covered and sealingly closed by the needle sheath. In such a configuration, the lumen of the needle shaft is fluid-tight with the exception of communication with the hub at the proximal portion of the needle shaft and communication with the lumen of the needle sheath through the side openings.

During use of the needle assembly, the distal portion of the needle shaft and a distal portion of the needle sheath will be positioned within tissue of a patient. Accordingly, a medical professional will be unable to visualize side openings without assistance from specialized imaging technology, such as ultrasound or radiography. Notably, however, proximal portions of the needle shaft and needle sheath will be positioned outside of the patient's body.

To aid in an operator knowing the number and/or area of side openings exposed in the distal end of the distal portion of the needle shaft, the needle may include indicia 250 near the proximal end of the needle sheath. Indicia may include at least a first mark 252. The first mark, when aligned with a corresponding portion of the needle sheath, such as the proximal most end of the needle sheath, can indicate to an operator that all side openings of the needle shaft are covered and sealingly closed by the needle sheath. Indicia 250 may also include marks 254, 256 and 258 that indicate different numbers and/or areas (e.g., lengths) of side openings that are exposed in the distal portion of the needle shaft (i.e., those uncovered by the needle sheath). Advantageously, allowing the operator to adjust the number and/or area of side openings exposed in the distal portion of the needle shaft can facilitate delivery of therapeutic agent to targeted tissue, such as abnormal tissue, without exposing healthy and/or untargeted tissue to the therapeutic agent.

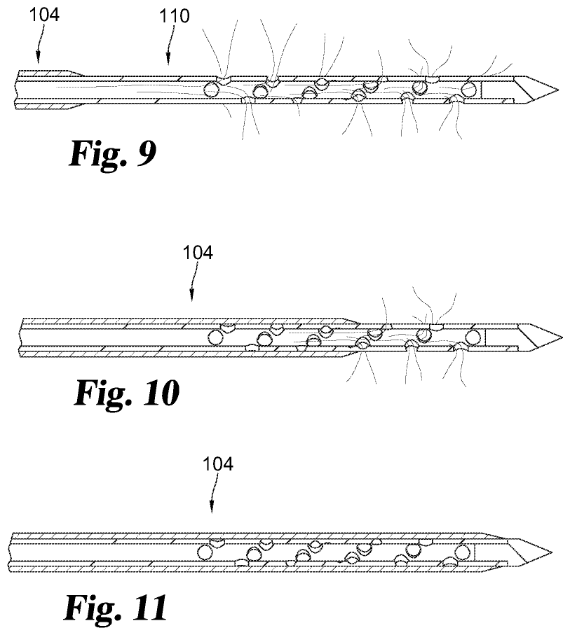

As shown in FIGS. 9-11, when the needle sheath is positioned over a side opening the needle sheath may sealingly close the side opening so as to prevent fluid from exiting the side opening and/or traveling distally and/or proximally between the needle sheath and the needle shaft. Additionally, by selectively covering side openings located near the proximal end of the distal section, an operator may increase the fluid pressure and/or velocity of fluid exiting the distally located side openings that are not covered by the needle sheath without having to increase the pressure and/or velocity of fluid entering the proximal portion of the needle shaft.

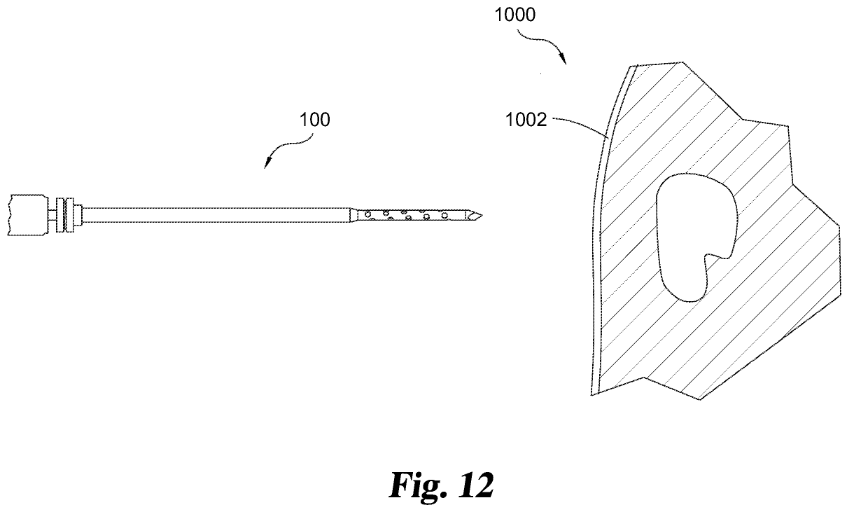

FIGS. 12-17 illustrate a method of using needle assemblies disclosed herein. As shown in FIG. 12, the needle assembly is initially located outside of the body of the patient 1000. In a provided configuration, the needle sheath may be retracted along the needle shaft to have one or more side openings of the plurality of side openings exposed in the distal portion of the needle shaft (shown in FIG. 12). Such an arrangement may be desirable to facilitate flushing of the needle lumen prior to insertion into the patient (e.g., flushing with saline).

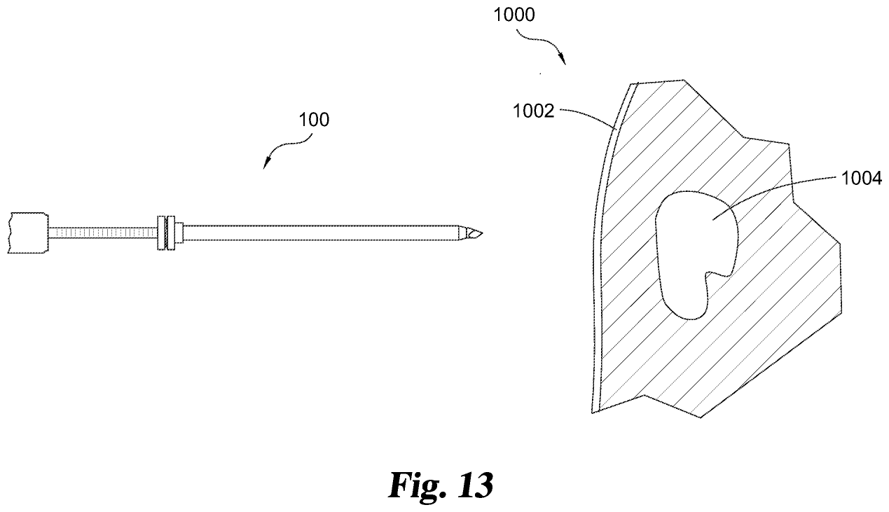

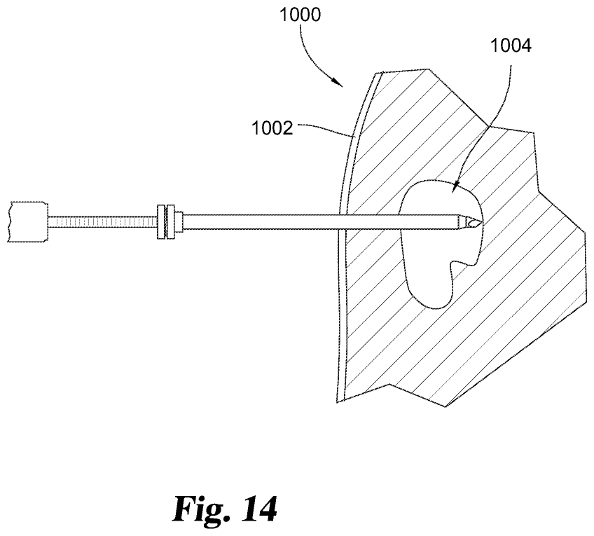

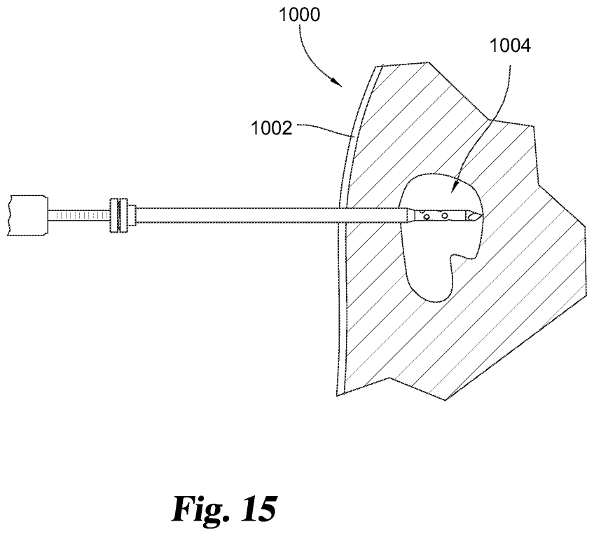

Prior to inserting the needle into the body of the patient (e.g., through the skin 1002 of the patient), the needle sheath may be advanced distally relative to the needle shaft so as to cover the side openings of the needle shaft, as shown in FIG. 13. With the needle sheath covering the side openings, the needle assembly is inserted into the body of the patient to a location wherein the portion of the needle containing side openings is positioned within the target area 1004, such as a tumor as shown in FIG. 14. With the side openings located within the target area, the needle sheath is retracted relative to the needle to expose one or more side openings located within the target area, as shown in FIG. 15. Preferably, the distal-most side opening is positioned within the target area and/or the needle sheath is retracted so that a proximal-most exposed side opening is also positioned within the target area. Preferably, no side openings of the needle sheath are exposed outside of the target area.

Preferably, the exposed side openings span at least 80% of the target area along a longitudinal axis of the needle. More preferably, the exposed side openings span at least 90% of the target area along a longitudinal axis of the needle

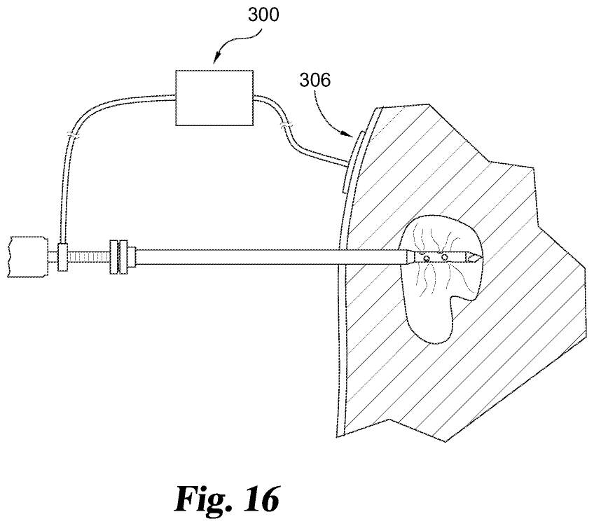

With the needle assembly in position and the desired area and number of side openings exposed within the target tissue, therapeutic agent may be infused through the lumen of the needle and out of the side openings into the target area, as shown in FIG. 16. Additionally, RF energy may be applied by an RF power supply 300 to the target area through use of one or more electrodes. For example, the RF power supply may be electrically coupled to the needle shaft and a surface electrode 306 so that the target area tissue may heated prior to, during, and/or after infusion of therapeutic agent. Preferably, the target area tissue is heated to a temperature of less than 45° Celsius to avoid ablation of target area tissue. It is believed that heating the target area tissue to a temperature of up-to but less than 45° Celsius can increase the cellular uptake of therapeutic agent delivered into the target area.

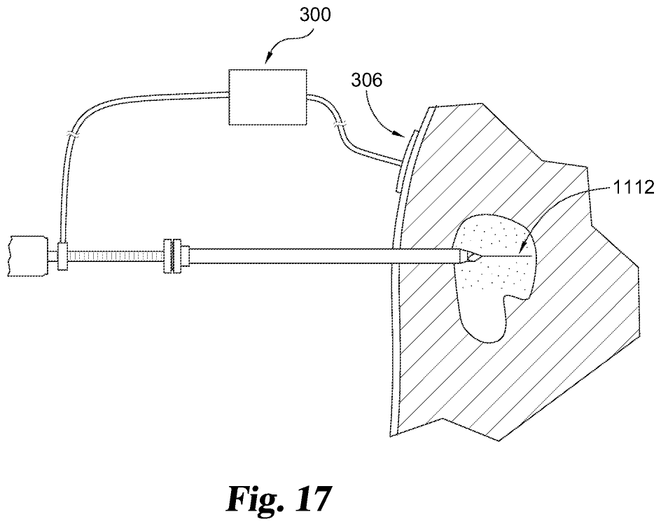

After delivery of therapeutic agent and/or RF power to the target area is complete, the needle shaft can be retracted relative to the needle sheath to sealingly cover and close the side openings, as shown in FIG. 17. An operator may then wait a period of time (e.g., one to five minutes) to allow the needle track 1112 within the target area tissue to close and the surrounding tissue to further uptake therapeutic agent that may have remained in the needle tract prior to withdrawing the needle assembly from the patient. Advantageously, this can aid in reducing therapeutic agent from leaking out of the target area into the surrounding tissue through the needle tract.

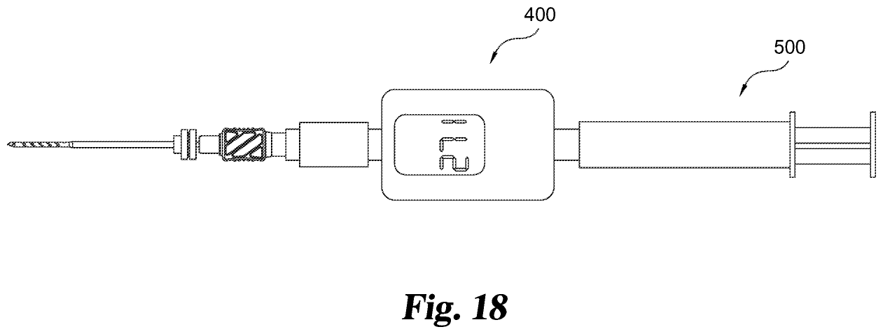

Turning now to FIG. 18, the needle assembly may include a pressure monitor 400 and/or an injector 500, each in fluid communication with the lumen of the needle. Preferably, the pressure monitor measures the pressure of fluid being infused from the injector into the lumen of the needle assembly. Advantageously, such arrangements can allow an operator to maintain the pressure below a threshold that may cause necrosis of certain therapeutic agent, e.g., cells.

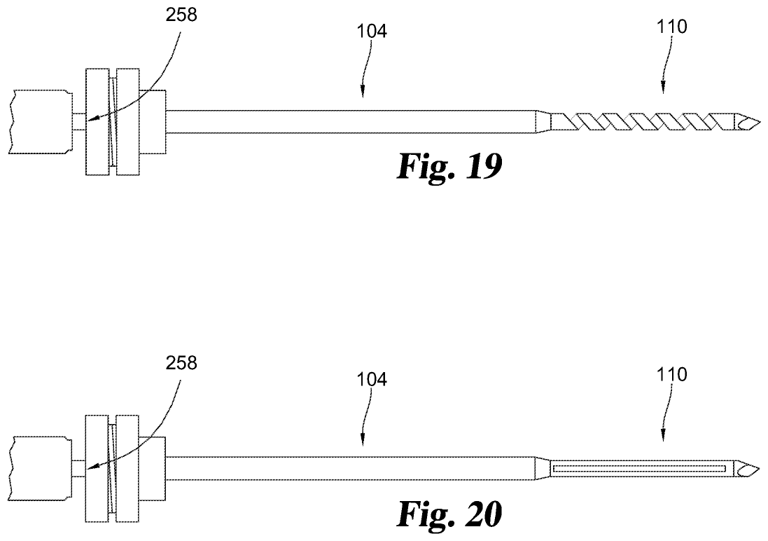

FIGS. 19 and 20 illustrate other needle arrangements envisioned for any of the above-mentioned assemblies and/or methods. FIG. 19 illustrates a needle assembly having an elongate side opening with a major axis that extends helically around the longitudinal axis of the needle shaft. FIG. 20 illustrates a needle assembly having an elongate side opening with a major axis extending parallel to the longitudinal axis of the needle shaft.

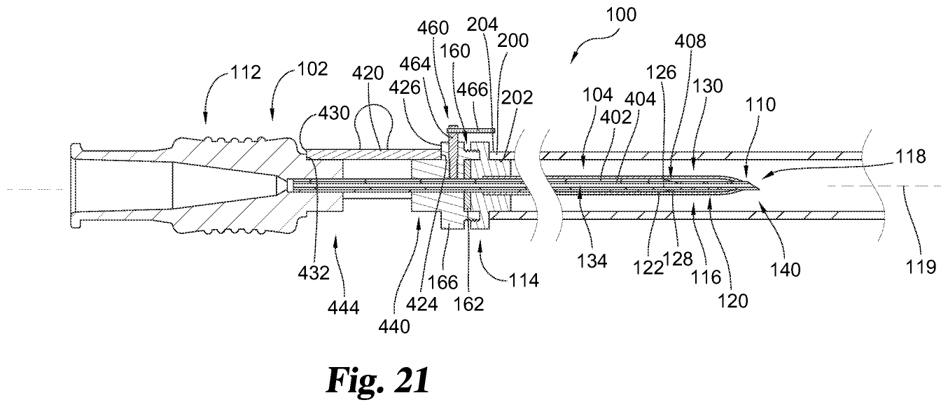

FIG. 21 illustrates an embodiment having a needle shaft having a first outer diameter along a first length and a second outer diameter along a second length. The first outer diameter can be defined by an outer surface of an outer sleeve 402 positioned around an inner needle 404 and longitudinally fixed thereto (e.g., soldered). The second outer diameter, in this instance, can be defined by an outer surface of the inner needle.

Between the first length and the second length is a shoulder 408 having a surface extending transverse to the outer surface of the first length and second length. The shoulder has an outer dimension (e.g., diameter) greater than that of the second length and, therefore, is believed to plug an end of a tissue tract created by the second length (e.g., when the shoulder contacts patient tissue) and/or plug an end of the needle sheath lumen to resist therapeutic agent from passing from the second length to the first length along the needle shaft. When positioned with in the needle sheath, the outer extent of the shoulder preferably fills the cross-sectional area of the needle sheath. And, in instances when the needle sheath is retracted such that the tapered distal portion of the needle sheath lies along the shoulder, the shoulder provides a greater surface area contacting the tapered inner surface of the sheath to provide greater resistance to fluid flowing proximally of the shoulder between the needle shaft and the needle sheath.

FIG. 21 also illustrates a spacer (e.g., a clip) 420 that retains the needle sheath in position relative to the needle shaft, and vice versa. The spacer has a distal-facing surface 424 that contacts a proximally-facing surface 426 of the hub of the needle sheath. And, the spacer has a proximally-facing surface 430 that contacts a distally-facing surface 432 of the hub of the needle (which is fixedly attached to the needle shaft). Accordingly, the column strength of the spacer resists the hub of the sheath from moving towards the hub of the needle.

The spacer can be removable from the hub of the needle sheath and/or the needle. Preferably, the spacer is made of a resilient material. The spacer can extend partially around a proximal portion 440 of the hub of the needle sheath and/or partially around a distal portion 444 of the hub of the needle such that the spacer can be “clipped” onto and/or off the hub(s).

FIG. 21 also illustrates a lock 460 for securing the needle sheath to the needle shaft at a desired location. In FIG. 21, the lock is illustrated as a setscrew 464 that extends through the hub of the needle sheath and contacts the needle shaft. The setscrew has a handle 466 to facilitate rotation of the setscrew using one's figures. The lock is preferably located proximally of the seal of the needle sheath.

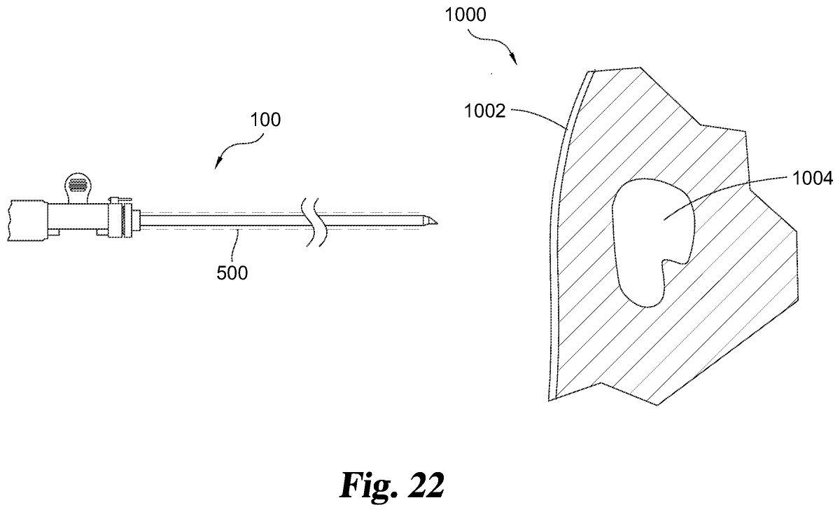

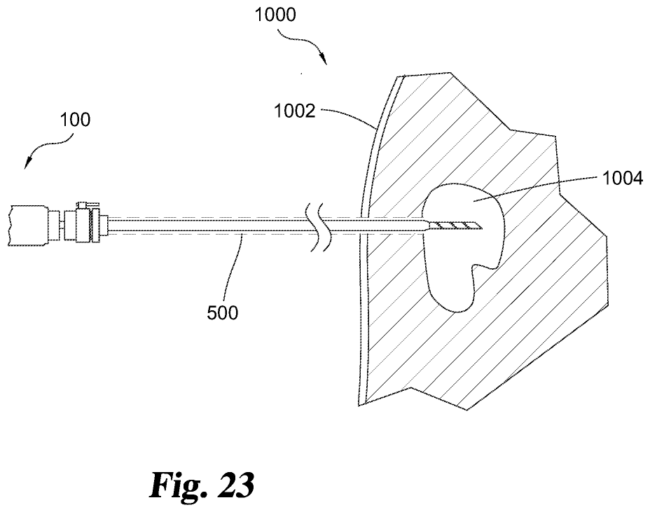

FIGS. 22 and 23 illustrate an exemplary method of inserting the needle into a target area. As shown in FIG. 22, the side openings of the needle shaft are covered by the needle sheath when the spacer is attached the needle and the needle sheath. With the spacer attached, the needle assembly can be advanced into patient tissue until the distal tip of the needle sheath is proximate the periphery of the target area. As with any of the embodiments disclosed herein, the advancement of the needle assembly can, optionally, be through an endoscope 500 (e.g., a catheter needle assembly). In such instances, the distal tip of the needle (e.g., a catheter needle) may be positioned within the needle sheath (e.g., a catheter) during advancement through the endoscope to prevent the distal tip from the needle from engaging (e.g., scraping and/or gouging) the lumen wall of the endoscope, which may dull the needle tip and/or damage the endoscope. When the distal tip of the needle sheath is in the desired location (e.g., beyond a distal tip of the endoscope), the spacer can be removed and the needle (i.e., needle shaft) can be advanced distally (relative to the needle sheath) into the target area to expose side openings in the target area. Preferably the distal tip of the needle sheath is against and/or in patient tissue when the needle is advanced distally so that the needle sheath distal tip closes (e.g., plugs) a proximal end of the tract formed in patient tissue.

Advantageously, after the side openings have been exposed in the target area, the lock may be engaged to secure the needle shaft relative to the needle sheath with the desired number/area (e.g., length) of side openings exposed within the target area. Accordingly, during the duration of the infusion, the needle sheath and needle shaft will be less likely to move relative to one another and, thereby, change the size of the infusion area. After infusion, and prior to withdrawal of the needle, the lock may be disengaged to allow subsequent withdrawal of the needle into the needle sheath.

After an infusion, the needle assembly may be withdrawn using any of the methods disclosed herein. For example, the needle may be withdrawn in an order reverse to that which it was inserted (e.g., withdrawing needle from the target area into the needle sheath while retaining the needle sheath in a stationary location in the patient tissue, attaching the spacer, and then withdrawing the entire assembly from the patient). Preferably, after the needle shaft is withdrawn at least partially into the needle sheath, the physician holds the distal tip of the needle shaft at a proximal end of the tract within the target area for one to five minutes in order to allow the needle tract in the target area to close, at least partially, and to reduce the volume of therapeutic agent in the needle tract which may follow the needle assembly through healthy patient tissue (i.e., the tissue adjacent the target area) during withdrawal of the needle assembly from the patient.

FIG. 23 illustrates a clamp lock 480 as an alternative to the setscrew arrangement of FIG. 21. A button 482 of the clamp lock may be actuatable by an operator (e.g., pressed) to apply and/or release at least some compressive force on the needle shaft. Preferably, applying compressive force resists translation and/or rotation of the needle sheath hub (and/or needle sheath) relative to the needle shaft and releasing compressive force allows such translation and/or rotation. The clamp lock may by biased (e.g., by a spring) into a configuration that exerts compressive force on the needle shaft. For example, the clamp lock may comprise the device of U.S. Pat. No. 4,453,292. The clamp lock may also comprise the device of U.S. Pat. No. 8,647,261.

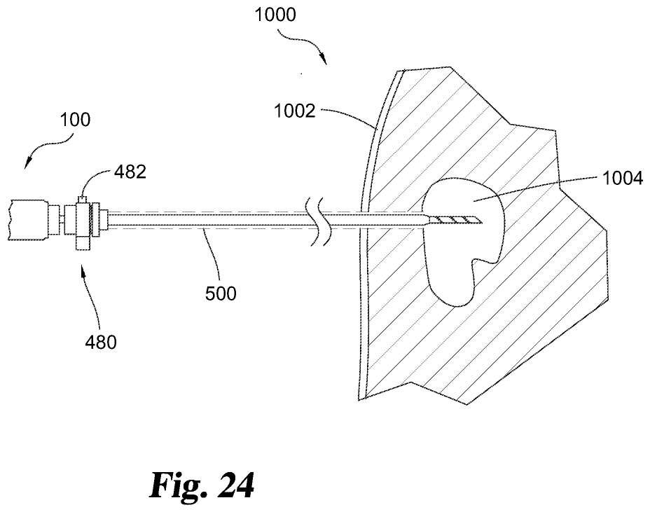

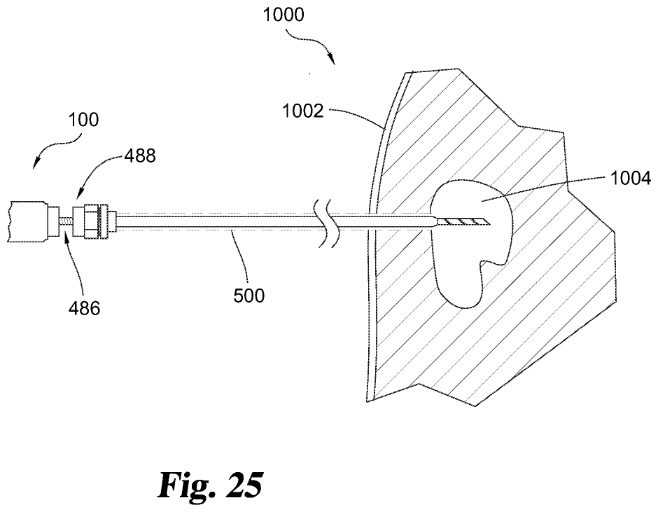

FIG. 24 illustrates an arrangement having a threaded needle shaft 486 and a floating nut 488 of the needle sheath hub engaging threads of the threaded needle shaft. Advantageously, such an arrangement can allow more precise movement of the needle sheath to relative to the needle shaft. Specifically, rotation of the floating nut relative to the needle shaft (or vice versa) can advance or retract the needle shaft relative to the needle sheath.

The following numbered clauses set out specific embodiments that may be useful in understanding the present invention:

1. A needle assembly, comprising:

a needle defining a lumen, the needle having a needle shaft extending from a hub;

the needle shaft having a proximal portion and a distal portion;

the distal portion having a distal tip and one or more side openings;

a needle sheath positioned around the needle shaft and slidable therealong, wherein the needle sheath is slidably configurable from a first configuration in which the needle sheath sealingly covers the one or more side openings to a second configuration in which the one or more side openings is/are at least partially uncovered by the needle sheath;

wherein the needle sheath has an interference fit around the needle shaft.

2. The needle assembly of clause 1, wherein the needle sheath includes a seal positioned around the needle shaft proximal of the one or more side openings and configured to prevent fluid from leaking between the needle sheath and the needle shaft and beyond a proximal end of the needle sheath.

3. The needle assembly of clause 2, wherein the seal comprises an o-ring positioned around the needle shaft.

4. The needle assembly of clause 2, wherein the seal comprises a tuohy borst valve.

5. The needle assembly of any preceding clause, wherein the assembly is effective to prevent fluid from leaking between the needle sheath and the needle shaft with a static fluid pressure of at least 50 mmHg in the lumen when the one or more side openings of the needle shaft is/are covered by the needle sheath and the needle assembly is in 1 atm.

6. The needle assembly of any preceding clause, wherein the assembly is effective to prevent fluid from leaking between the needle sheath and the needle shaft with a static fluid pressure of at least 250 mmHg in the lumen when the one or more side openings of the needle shaft is/are covered by the needle sheath and the needle assembly is in 1 atm.

7. The needle assembly of any preceding clause, wherein the proximal portion of the needle shaft includes indicia including a first mark adjacent to a second mark;

wherein when the needle sheath is in the first configuration, a proximal portion of the needle sheath is aligned with the first mark; and

wherein when the needle sheath is in the second configuration, the proximal portion of the needle sheath is aligned with the second mark.

8. The needle assembly of clause 7, wherein the one or more side openings is a first side opening of the needle shaft; and wherein the first mark is spaced from the second mark a distance equal to spacing between a distal-most edge of the first side opening and a distal-most edge of an adjacent second side opening of the needle shaft.

9. The needle assembly of any preceding clause, comprising a pressure monitor coupled to the needle and configured to measure pressure of fluid entering the lumen.

10. The needle assembly of any preceding clause, comprising an injector configured to inject fluid into the lumen.

11. The needle assembly of any preceding clause, comprising an RF power supply and a ground electrode, wherein the RF power supply is in electrical communication with the needle shaft and the ground electrode and is configured to generate a voltage difference between the needle shaft and the ground electrode.

12. The needle assembly of any preceding clause, wherein the distal tip is a closed, tissue-penetrating tip.

13. The needle assembly of any preceding clause, wherein the lumen is free of a movable tube positioned therein.

14. The needle assembly of any preceding clause, comprising a protective cover positioned around the needle sheath and the needle shaft with the needle sheath positioned around the needle shaft.

15. A kit comprising the needle assembly of any preceding clause and a volume of liquid containing a suspension of cells therein.

16. A method of infusing a therapeutic agent into a volume of tissue, comprising:

i. inserting a distal portion of a needle assembly into the volume of tissue in a first configuration, the needle assembly comprising a needle shaft and a needle sheath, the needle shaft having a length, a distal tip, and one or more side openings in the the distal portion, the needle sheath slidably positioned around the needle shaft, the needle sheath configurable from the first configuration in which the needle sheath sealingly covers the one or more side openings to a second configuration in which the one or more side openings is/are at least partially uncovered;

ii. configuring the needle assembly into the second configuration within the volume of tissue;

iii. infusing a therapeutic through the needle shaft in the second configuration and out of the exposed one or more side openings into the volume of tissue;

iv. configuring the needle assembly into the first configuration within the volume of tissue by withdrawing the needle shaft relative to the needle sheath such that the needle shaft moves a greater distance relative to the volume of tissue than the needle sheath; and

v. retracting the needle shaft in the first configuration from the volume of tissue.

17. The method of clause 16, wherein configuring the needle assembly into the second configuration comprises withdrawing the needle sheath relative to the needle shaft such that the needle sheath moves a greater distance relative to the volume of tissue than the needle shaft.

18. The method of clause 16, wherein configuring the needle assembly into the second configuration comprises advancing the needle shaft from the needle sheath such that the needle shaft moves a greater distance relative to the volume of tissue than the needle sheath.

19. The method of clause 16, 17, or 18, wherein the one or more side openings is a first side opening of the needle shaft; and

wherein in the second configuration a second side opening is positioned proximally of the first side opening and is sealingly covered by the needle sheath.

20. The method of clause 16, 17, 18, or 19, comprising applying RF energy to the volume of tissue from the needle shaft in the second configuration.

21. A needle assembly, comprising:

a needle defining a lumen, the needle having a needle shaft extending from a hub;

the needle shaft having a proximal portion and a distal portion;

the distal portion having a distal tip and one or more side openings spaced along a length of the distal portion; and

a needle sheath slidably positioned around the needle shaft, wherein the needle sheath is configurable from a first configuration in which the needle sheath sealingly covers the one or more side openings to a second configuration in which the one or more side openings is/are at least partially uncovered;

wherein the needle sheath is effective to prevent fluid from leaking from the one or more side openings with a static fluid pressure of at least 50 mmHg in the lumen when the one or more side openings is/are covered by the needle sheath and the needle assembly is in 1 atm.

22. The needle assembly of clause 21, wherein the needle sheath is effective to prevent fluid from leaking from the one or more side openings with a static fluid pressure of at least 250 mmHg in the lumen when the one or more side openings is/are covered by the needle sheath and the needle assembly is in 1 atm.

23. The needle assembly of clause 21 or 22, comprising a seal positioned around the needle shaft proximal of the one or more side openings and configured to prevent fluid from leaking between the needle sheath and the needle shaft and beyond a proximal end of the needle sheath.

24. The needle assembly of clause 23, wherein the seal comprises an o-ring positioned around the needle shaft.

25. The needle assembly of clause 23, wherein the seal comprises a tuohy borst valve.

26. The needle assembly of any one of clauses 23-25, wherein the seal is effective to prevent fluid from leaking between the needle sheath and the needle shaft with a static fluid pressure of at least 50 mmHg in the lumen when the one or more side openings is/are covered by the needle sheath and the needle assembly is in 1 atm.

27. The needle assembly of any one of clauses 23-25, wherein the seal is effective to prevent fluid from leaking between the needle sheath and the needle shaft with a static fluid pressure of at least 250 mmHg in the lumen when the one or more side openings is/are covered by the needle sheath and the needle assembly is in 1 atm.

28. The needle assembly of any one of clauses 21-27, wherein the proximal portion of the needle shaft includes indicia including a first mark spaced from a second mark.

29. The needle assembly of clause 27, wherein when the needle sheath is in the first configuration, a proximal portion of the needle sheath is aligned with the first mark.

30. The needle assembly of clause 27 or 28, wherein when the needle sheath is in the second configuration, the proximal portion of the needle sheath is aligned with the second mark.

31. The needle assembly of any one of clauses 21-30, comprising a pressure monitor coupled to the needle and configured to measure pressure of fluid entering the lumen.

32. The needle assembly of any one of clauses 21-31, comprising an injector configured to inject fluid into the lumen.

33. The needle assembly of clause 21, comprising an RF power supply and a ground electrode, wherein the RF power supply is in electrical communication with the needle shaft and the ground electrode and is configured to generate a voltage difference between the two.

34. The needle assembly of clause 21, wherein the distal tip is a tissue penetrating tip free of an opening.

35. The needle assembly of clause 21, wherein the lumen is free of a tube positioned therein.

36. The needle assembly of clause 21, wherein the one or more side openings is a first side opening and the needle shaft has a second side opening; and wherein the first side opening and the second side opening are spaced from one another around a circumference of the needle shaft.

37. A kit comprising the needle assembly of clause 21 and a volume of liquid containing a suspension of cells therein.

38. A needle, comprising:

a needle hub and a needle shaft;

the needle shaft having a first outer diameter along a first length and a second outer diameter along a second length, the first outer diameter being greater than the second outer diameter, the second length being proximal of a tissue penetrating needle tip, and the first length being proximal of the second length; and

the needle shaft having a plurality of side openings along said second length.

39. The needle of clause 38, wherein the needle shaft has an inner surface defining a lumen;

wherein the needle shaft has a wall thickness measured between the inner surface and the outer surface; and

wherein the wall thickness in said first length is greater than the wall thickness in said second length.

40. The needle of clause 39, wherein the needle shaft includes an outer sleeve positioned around an inner needle along said first length, the outer sleeve longitudinally fixed to said inner needle;

said outer sleeve and inner needle defining said wall thickness.

41. The needle of clause 40, wherein the outer sleeve is soldered to the inner needle.

42. An assembly comprising the needle of any one of clauses 38-41 and a needle sheath positioned around the needle shaft and having a length less than the needle shaft.

43. The assembly of clause 42, wherein the needle sheath has an inner surface defining a needle sheath lumen, and wherein the inner surface tapers inwardly in a distal portion of the needle sheath.

44. An assembly comprising an endoscope and the needle assembly of any preceding clause having a length sufficient to extend through a lumen of the endoscope.

While the invention has been illustrated and described in detail in the drawings and foregoing description, the same is to be considered as illustrative and not restrictive in character, it being understood that only the preferred embodiment has been shown and described and that all changes, equivalents, and modifications that come within the spirit of the inventions defined by following claims are desired to be protected. All publications, patents, and patent applications cited in this specification are herein incorporated by reference as if each individual publication, patent, or patent application were specifically and individually indicated to be incorporated by reference and set forth in its entirety herein.

The language used in the claims and the written description is to only have its plain and ordinary meaning, except for terms explicitly defined below. Such plain and ordinary meaning is defined here as inclusive of all consistent dictionary definitions from the most recently published (on the filing date of this document) general purpose Merriam-Webster dictionary.

As used in the claims and the specification, the following terms have the following defined meanings:

The term “and/or” is inclusive here, meaning “and” as well as “or”. For example, “P and/or Q” encompasses, P, Q, and P with Q; and, such “P and/or Q” may include other elements as well.

The term “cells” as used herein refers to a microscopic mass of protoplasm bounded externally by a semipermeable membrane, usually including one or more nuclei and various other organelles. Use of the term “cells” in this disclosure includes cells suspended in fluid media (e.g., heparinized saline). For example, the cells/cellular material may be T-cells. The cells may be provided by a manufacturer preloaded in the device, or in a separate container or arrangement to be incorporated into or onto the delivery device prior to or during the cell delivery procedure. During infusion, the cells can be loaded into the needle assembly followed by flushing fluid (saline, media, etc.).

The terms “comprise”, “comprises”, “comprised” or “comprising” as used herein (including the claims) are to be interpreted as specifying the presence of the stated features, integers, steps or components, but not precluding the presence of one or more other features, integers, steps or components, or group thereof.

The term “coupled” as used herein has the meaning, joined or linked directly or indirectly.

The term “distal” as used herein refers to the opposite of “proximal” (i.e., the patient end/treating end).

The term “electrode” as used herein refers to an electrical conductor used to establish electrical contact.

The term “hub” as used herein refers to the expanded portion of a hollow needle that serves as a handle for manipulation. The “hub” often includes at least one opening communicating with an interior lumen of the needle. Such openings may include sites for attachment of a syringe, infusion tube, and/or other appliance. For example, the openings may include a connector, such as a male or female Luer connector.

The term “interference fit” as used herein has the meaning a fit between parts in which the external dimension of a first part exceeds the internal dimension of a second part into which the first part fits.

The term “lumen” as used herein refers to the bore of a tube (as of a hollow needle or catheter).

The term “needle” as used herein refers to a slender hollow instrument for introducing material into or removing material from the body (as by insertion under the skin). Such “needles” are often formed of metal (e.g., stainless steel).

The term “patient” as used herein includes an individual awaiting or under medical care and treatment. The term is includes those of both human and veterinary nature.

The term “proximal” as used herein refers to an end or direction associated with a physician or other treating personnel during a device operation.

The term “power supply” as used herein refers to a device providing electrical power, including but not limited to, electrical power at a frequency between 350 kHz and 500 kHz.

The term “pressure monitor” as used herein refers to device capable of measuring fluid pressure, static pressure and/or dynamic pressure. The term includes, but is not limited, the electromechanical transducers.

The term “seal” as used herein refers to a member for preventing fluid leakage, such as an o-ring. The term can also include septums and valves, such as a tuohy borst valve, when arranged to prevent fluid leakage.

The term “side opening” as used herein refers to an opening in a side of the needle shaft. Such openings can include holes and slots. The opening(s) can be circular and/or polygonal in cross-section. The openings may be elongate (e.g., oblong). For example, the opening(s) may be oval or obround. The opening(s) may be space between coils, such as between coils of a helically-coiled wire defining a tube. The terms “side opening” and “side openings” as used herein may be a subset of a plurality of side openings of the needle shaft. For example, the term “side openings” may be a first group of a plurality of side openings. The plurality of side openings may include a second group of side openings. The second group of side openings can be positioned proximally of the first group of side openings.

The term “sheath” as used herein refers to a generally to a cover and includes hollow cylindrical members. Such members may be formed of a metal and/or polymeric material.

The term “slidable” as used herein can include longitudinally slidable and/or rotationally slidable.

The term “target area” as used herein refers to the volume in the patient intended to receive therapy, such as a lesion. Target areas can be malignant and/or benign. Exemplary target areas include tumors, polyps, and abscesses.

The term “therapeutic agent” as used herein means a substance useful in the treatment of a disease or disorder. It includes, but is not limited to small molecule drugs and contrast agents, nanoparticles, macromolecules, and cells. The term includes small molecule drugs useful for localized chemotherapy/oncology and/or vascular intervention such as dissolving thrombus and/or reducing vascular calcification. For example, drugs such as paclitaxel, rapamycin, myotropic/neurotropic antispasmodics, and anticalcificants such as phosphate binders are included. Contrast agents suitable for MRI, X-Ray, and/or ultrasound imaging are included, such as gadolinium, manganese, iron oxide, and iodine-based (ionic/non-ionic) contrast agents. Organic, inorganic, and/or complex/polymeric nanoparticles useful for thermal ablation and targeted drug-delivery are contemplated. This includes but is not limited to liposomes, micelles, perfluorocarbons, gold nanoparticles, superparamagnetic iron oxide nanoparticles (SPION), dendrimers and functionalized nanoparticles. Macromolecule proteins, peptides, and/or synthetic polymers useful for biochemical thrombectomy, cell adhesion, coercive morphogenesis, prolonged drug-release, and/or sealants are contemplated. This includes but is not limited to fibrinolytics (e.g., urokinase, tPA), adhesional proteins (e.g., Fn, Lama, Col), growth factors (e.g., VEGF, TGF, Insulin), drug-eluting gels, hydrogels and glues. Environmentally-responsive hydrogels that can transition from liquid to gel form at a desired temperature (e.g., at 37° C.) and concentration are contemplated. Cells including differentiated, stem/progenitor, and/or genetically modified cells useful for re-endothelialization, endothelial regeneration and/or cellular therapy are contemplated as well as antisense and monoclonal antibodies.

The term “tip” as used herein refers to an end-most point.

It will be understood that “syringe” and “needle” are exemplary modes, and substitutes for these components or alternative structures may be used in specific embodiments. For example, devices such as pumps, power injectors, indeflators, compressible bladders, and the like may all be used as a substitute for a syringe. Furthermore, the term “needle” may include hypodermic needles, cannulas, microneedles, and nanoneedles.

1. A needle assembly, comprising:
a needle defining a lumen, the needle having a needle shaft extending from a hub;
the needle shaft having a proximal portion and a distal portion;
the distal portion having a distal tip and one or more side openings;
a needle sheath positioned around the needle shaft and slidable therealong, wherein the needle sheath is slidably configurable from a first configuration in which the needle sheath sealingly covers the one or more side openings to a second configuration in which the one or more side openings is/are at least partially uncovered by the needle sheath; and
wherein the needle sheath has an interference fit around the needle shaft.

2. The needle assembly of claim 1, wherein the needle sheath includes a seal positioned around the needle shaft proximal of the one or more side openings and configured to prevent fluid from leaking between the needle sheath and the needle shaft and beyond a proximal end of the needle sheath.

3. The needle assembly of claim 2, wherein the seal comprises an o-ring positioned around the needle shaft.

4. The needle assembly of claim 2, wherein the seal comprises a tuohy borst valve.

5. The needle assembly of claim 2, wherein the assembly is effective to prevent fluid from leaking between the needle sheath and the needle shaft with a static fluid pressure of at least 50 mmHg in the lumen when the one or more side openings of the needle shaft is/are covered by the needle sheath and the needle assembly is in 1 atm.

6. The needle assembly of claim 2, wherein the assembly is effective to prevent fluid from leaking between the needle sheath and the needle shaft with a static fluid pressure of at least 250 mmHg in the lumen when the one or more side openings of the needle shaft is/are covered by the needle sheath and the needle assembly is in 1 atm.

7. The needle assembly of claim 2, wherein the proximal portion of the needle shaft includes indicia including a first mark adjacent to a second mark;
wherein when the needle sheath is in the first configuration, a proximal portion of the needle sheath is aligned with the first mark; and
wherein when the needle sheath is in the second configuration, the proximal portion of the needle sheath is aligned with the second mark.

8. The needle assembly of claim 7, wherein the one or more side openings is a first side opening of the needle shaft; and
wherein the first mark is spaced from the second mark a distance equal to spacing between a distal-most edge of the first side opening and a distal-most edge of an adjacent second side opening of the needle shaft.

9. The needle assembly of claim 1, comprising a pressure monitor coupled to the needle and configured to measure pressure of fluid entering the lumen.

10. The needle assembly of claim 1, comprising an injector configured to inject fluid into the lumen.

11. The needle assembly of claim 1, comprising an RF power supply and a ground electrode, wherein the RF power supply is in electrical communication with the needle shaft and the ground electrode and is configured to generate a voltage difference between the needle shaft and the ground electrode.

12. The needle assembly of claim 1, wherein the distal tip is a closed, tissue-penetrating tip.

13. The needle assembly of claim 1, wherein the lumen is free of a movable tube positioned therein.

14. The needle assembly of claim 1, comprising a protective cover positioned around the needle sheath and the needle shaft with the needle sheath positioned around the needle shaft.

15. A kit comprising the needle assembly of claim 1 and a volume of liquid containing a suspension of cells therein.

16. A method of infusing a therapeutic agent into a volume of tissue, comprising:
i. inserting a distal portion of a needle assembly into the volume of tissue in a first configuration, the needle assembly comprising a needle shaft and a needle sheath, the needle shaft having a length, a distal tip, and one or more side openings in the the distal portion, the needle sheath slidably positioned around the needle shaft, the needle sheath configurable from the first configuration in which the needle sheath sealingly covers the one or more side openings to a second configuration in which the one or more side openings is/are at least partially uncovered;
ii. configuring the needle assembly into the second configuration within the volume of tissue;
iii. infusing a therapeutic through the needle shaft in the second configuration and out of the exposed one or more side openings into the volume of tissue;
iv. configuring the needle assembly into the first configuration within the volume of tissue by withdrawing the needle shaft relative to the needle sheath such that the needle shaft moves a greater distance relative to the volume of tissue than the needle sheath; and
v. retracting the needle shaft in the first configuration from the volume of tissue.

17. The method of claim 16, wherein configuring the needle assembly into the second configuration comprises withdrawing the needle sheath relative to the needle shaft such that the needle sheath moves a greater distance relative to the volume of tissue than the needle shaft.

18. The method of claim 16, wherein configuring the needle assembly into the second configuration comprises advancing the needle shaft from the needle sheath such that the needle shaft moves a greater distance relative to the volume of tissue than the needle sheath.

19. The method of claim 16, wherein the one or more side openings is a first side opening of the needle shaft; and
wherein in the second configuration a second side opening is positioned proximally of the first side opening and is sealingly covered by the needle sheath.

20. The method of claim 16, comprising applying RF energy to the volume of tissue from the needle shaft in the second configuration.
